(19) United States
(12) Patent Application Publication
(10) Pub. No.: US 2026/0115007 A1
(43) Pub. Date: Apr. 30, 2026

(54) ADJUNCTIVE IMPLANTS FOR FRACTURE STABILIZATION

(71) Applicant: PZ Ortho, Pocatello, ID (US)

(72) Inventors: Edward PEREZ, Ft. Lauderdale, FL (US); Nephi ZUFELT, Pocatello, ID (US)

(21) Appl. No.: 19/048,769

(22) Filed: Feb. 7, 2025

Related U.S. Application Data
(60) Provisional application No. 63/550,895, filed on Feb. 7, 2024.

(51) Int. Cl. A61F 2/46; A61B 17/80; A61F 2/28; A61F 2/30; A61F 2/40; A61L 27/58

(52) U.S. Cl. CPC A61F 2/4601; A61B 17/8061; A61F 2/4003; A61L 27/58; A61F 2002/2835; A61F 2002/30062; A61F 2002/30581; A61F 2002/30736; A61F 2002/30985; A61L 2430/02

(57) ABSTRACT

A system for stabilization of a bone of a patient, the bone including a fracture and a bone void, and the bone void resulting in insufficient structural strength, may include a first implant configured to reduce the fracture of the bone, and an adjunctive implant configured to be coupled with the first implant. The adjunctive implant may be further configured to be received within the bone void such that the adjunctive implant may provide structural support to the bone from within the bone void.

### CROSS-REFERENCE TO RELATED APPLICATIONS

The present application claims the benefit of U.S. Provisional Application No. 63/550,895 filed on Feb. 7, 2024, entitled ADJUNCTIVE IMPLANTS FOR FRACTURE STABILIZATION, which is incorporated by reference as though set forth herein in its entirety.

### TECHNICAL FIELD

The present disclosure relates to systems and methods for use in orthopedic surgery. More specifically, the present disclosure relates to adjunctive implants for fracture stabilization in orthopedic surgery.

### BACKGROUND

Bone fractures, particularly those involving complex anatomical sites such as the trochanter, proximal humerus, wrist, or distal femur, present significant challenges in orthopedic treatment. The primary goal of fracture repair is to stabilize the bone to promote proper healing, restore function, and minimize complications. Current methods of internal fixation often rely on devices such as nails, pins, screws, and plates. While these solutions have been widely adopted, they are not without limitations, especially in cases involving comminuted fractures, osteoporotic bone, or fractures with substantial biomechanical demands.

One key limitation of traditional fixation methods is the lack of sufficient structural support in bones. Nails, pins, and screws rely heavily on the mechanical strength of the bone to maintain stability, and in cases where the bone is compromised, fixation devices may loosen, migrate, stress shield, or fail altogether. This is particularly problematic in fractures of the proximal humerus, where the cancellous bone offers limited purchase for screws, leading to complications such as screw penetration into the joint or loss of fracture reduction.

Fractures of the wrist, particularly distal radius fractures, also pose unique challenges. Although commonly treated with volar plates or external fixation, these methods may not provide sufficient support in multifragmentary fractures or cases with severe bone loss. The limited stability can compromise early mobilization, resulting in stiffness or suboptimal functional outcomes.

To address these challenges, there is a need for an adjunctive implant that complements traditional fixation devices by providing additional structural support. Such an implant should be capable of distributing loads more effectively, reducing the reliance on bone quality, and enhancing stability in complex or compromised fractures. The development of an innovative adjunctive implant could improve clinical outcomes, reduce the risk of complications, and facilitate faster recovery in patients with difficult-to-treat fractures.

### SUMMARY

The various systems and methods of the present disclosure have been developed in response to the present state of the art, and in particular, in response to the problems and needs in the art that have not yet been fully solved by currently available fracture repair systems and methods.

In some embodiments, a system for stabilization of a bone of a patient, the bone including a fracture and a bone void, and the bone void resulting in insufficient structural strength, may include a first implant configured to reduce the fracture of the bone, and an adjunctive implant configured to be coupled with the first implant. The adjunctive implant may be further configured to be received within the bone void such that the adjunctive implant may provide structural support to the bone from within the bone void.

In the system of any preceding paragraph, the adjunctive implant may further include a bioabsorbable material configured to be absorbed by the patient.

In the system of any preceding paragraph, the adjunctive implant may include a malleable silk balloon configured to receive a bone filler material.

In the system of any preceding paragraph, the adjunctive implant may include a compressed state and an expanded state, wherein, in the expanded state, the adjunctive implant may generally conform to a shape of the bone void.

In the system of any preceding paragraph, the system may further include a cannula configured to receive the adjunctive implant in the compressed state, wherein, with the adjunctive implant in the compressed state, the adjunctive implant may be insertable through the cannula into the bone void.

In the system of any preceding paragraph, the system may further include a bone filler material configured to be received within the adjunctive implant, the adjunctive implant may further include an interior portion, and the adjunctive implant may be transitionable from the compressed state to the expanded state through introduction of the bone filler material into the interior portion.

In the system of any preceding paragraph, the first implant and the adjunctive implant may be additively manufactured as a unified implant.

In some embodiments, a system for stabilization of a bone of a patient, the bone including a fracture and a bone void, may include a first implant configured to reduce the fracture of the bone, the first implant having an access aperture, an adjunctive implant having a compressed state and an expanded state, and a bone filler material configured to be introduced into the adjunctive implant. The adjunctive implant may be insertable through the access aperture into the bone void, the bone filler material may be insertable through the access aperture into the adjunctive implant, and the adjunctive implant may be transitionable from the compressed state to the expanded state through introduction of the bone filler material.

In the system of any preceding paragraph, with the adjunctive implant in the expanded state, the adjunctive implant may provide structural support to the bone.

In the system of any preceding paragraph, the adjunctive implant may be configured to be coupled with the first implant.

In the system of any preceding paragraph, the adjunctive implant may further include a bioabsorbable material configured to be absorbed by the patient.

In the system of any preceding paragraph, the system may further include a cannula insertable through the access aperture and configured to receive the adjunctive implant in the compressed state. With the adjunctive implant in the compressed state, the adjunctive implant may be insertable through the cannula into the bone void.

In the system of any preceding paragraph, the bone filler material may be insertable through the cannula into the adjunctive implant.

In the system of any preceding paragraph, with the adjunctive implant in the expanded state, the adjunctive implant may generally conform to a shape of the bone void.

In some embodiments, a system for stabilization of a bone of a patient, the bone including a fracture and a bone void, may include a first segment configured to reduce the fracture of the bone, and a second segment configured to be coupled with the first segment. The first segment and the second segment may be additively manufactured, the first segment may include a first material, the second segment may include a second material, different than the first material, and the first material may be more rigid than the second material.

In the system of any preceding paragraph, at least one of the first material and the second material may include a bioabsorbable material configured to be absorbed by the patient.

In the system of any preceding paragraph, at least one of the first segment and the second segment may include a surface coating configured to promote osteointegration.

In the system of any preceding paragraph, the system may be configured to stabilize the fracture and the bone void of a humerus.

In the system of any preceding paragraph, the second segment may be configured to be received within the bone void.

In the system of any preceding paragraph, the system may further include a bone filler material configured to be received within the second segment, and the second segment may be transitionable from a compressed state to an expanded state through introduction of the bone filler material.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings.

FIG. 1 is a perspective view of some of the bones generally forming a shoulder joint;

FIG. 2 is a perspective view of an exemplary shoulder joint with an exemplary fracture of a proximal humerus;

FIG. 3 is a perspective view of an exemplary shoulder joint with an exemplary fracture of a proximal humerus and a bone plate;

FIG. 4 is a perspective view of an exemplary shoulder joint with an exemplary fracture of a proximal humerus, a bone plate, and a cannula according to an embodiment of the present disclosure;

FIG. 5 is a perspective view of an exemplary shoulder joint with an exemplary fracture of a proximal humerus, a bone plate, a cannula, and an adjunctive implant according to an embodiment of the present disclosure;

FIG. 6 is a perspective view of an exemplary shoulder joint with an exemplary fracture of a proximal humerus, a bone plate, a cannula, and the adjunctive implant of FIG. 5;

FIG. 7 is a perspective view of an exemplary femur with an exemplary fracture and bone fragment;

FIG. 8 is a perspective view of an exemplary femur with an exemplary fracture and bone fragment and an exemplary intramedullary nail and fastener;

FIG. 9 is a perspective view of an exemplary femur with an exemplary fracture and bone fragment, an exemplary intramedullary nail and fastener, and a cannula according to an embodiment of the present disclosure;

FIG. 10 is a perspective view of an exemplary femur with an exemplary fracture and bone fragment, an exemplary intramedullary nail and fastener, a cannula, and an adjunctive implant according to an embodiment of the present disclosure;

FIG. 11 is a perspective view of an exemplary femur with an exemplary fracture and bone fragment, an exemplary intramedullary nail and fastener, a cannula, and the adjunctive implant of FIG. 10;

FIG. 12 is a perspective view of an exemplary femur with an exemplary fracture and bone fragment, an exemplary intramedullary nail and fastener, and the adjunctive implant of FIG. 10;

FIG. 13 is a perspective view of an exemplary femur with an exemplary fracture and bone fragment, a bone plate, and an adjunctive implant according to an embodiment of the present disclosure; and

FIG. 14 is a perspective view of some of the bones generally forming a wrist, pins, and an adjunctive implant according to an embodiment of the present disclosure.

FIG. 15A is a side view of a bone plate according to an embodiment of the present disclosure.

FIG. 15B is a front view of the bone plate of FIG. 15A.

FIG. 16A is a side view of a bone plate according to an embodiment of the present disclosure.

FIG. 16B is a front view of the bone plate of FIG. 16A.

FIG. 17A is a side view of a bone plate according to an embodiment of the present disclosure.

FIG. 17B is a front view of the bone plate of FIG. 17A.

FIG. 18A is a side view of a bone plate according to an embodiment of the present disclosure.

FIG. 18B is a front view of the bone plate of FIG. 18A.

FIG. 19A is a perspective view of a bone plate according to an embodiment of the present disclosure.

FIG. 19B is a side view of the bone plate of FIG. 19A.

FIG. 19C is a front view of the bone plate of FIG. 19A.

FIG. 20A is a front view of an expandable adjunctive implant in a compressed state according to an embodiment of the present disclosure.

FIG. 20B is a perspective view of the expandable adjunctive implant of FIG. 20A in the compressed state.

FIG. 21A is a front view of the expandable adjunctive implant of FIG. 20A in an expanded state according to an embodiment of the present disclosure.

FIG. 21B is a perspective view of the expandable adjunctive implant of FIG. 21A in the expanded state.

FIG. 22A is a side view of a system for stabilization of a bone of a patient including the expandable adjunctive implant of FIG. 20A and the bone plate of FIG. 16A with the expandable adjunctive implant in a compressed state according to an embodiment of the present disclosure.

FIG. 22B is a perspective view of the system for stabilization of a bone of a patient of FIG. 22A in the compressed state.

FIG. 23A is a side view of a system for stabilization of a bone of a patient including the expandable adjunctive implant of FIG. 20A and the bone plate of FIG. 16A with the expandable adjunctive implant in an expanded state according to an embodiment of the present disclosure.

FIG. 23B is a perspective view of the system for stabilization of a bone of a patient of FIG. 23A in the expanded state.

FIG. 24A is a side view of a system for stabilization of a bone of a patient including the expandable adjunctive implant of FIG. 20A and the bone plate of FIG. 19A with the expandable adjunctive implant in a compressed state according to an embodiment of the present disclosure.

FIG. 24B is a perspective view of the system for stabilization of a bone of a patient of FIG. 24A in the compressed state.

FIG. 25A is a side view of a system for stabilization of a bone of a patient including the expandable adjunctive implant of FIG. 20A and the bone plate of FIG. 19A with the expandable adjunctive implant in an expanded state according to an embodiment of the present disclosure.

FIG. 25B is a perspective view of the system for stabilization of a bone of a patient of FIG. 25A in the expanded state.

FIG. 26A is a side view of a system for stabilization of a bone of a patient including the expandable adjunctive implant of FIG. 20A and the bone plate of FIG. 19A with the expandable adjunctive implant in a compressed state and the system in a pre-coupled state according to an embodiment of the present disclosure.

FIG. 26B is a perspective view of the system for stabilization of a bone of a patient of FIG. 26A in the pre-coupled state.

FIG. 27A is a side view of a system for stabilization of a bone of a patient including the expandable adjunctive implant of FIG. 20A and the bone plate of FIG. 19A with the expandable adjunctive implant in a compressed state and the system in a pre-coupled state according to an embodiment of the present disclosure.

FIG. 27B is a perspective view of the system for stabilization of a bone of a patient of FIG. 27A in the pre-coupled state.

It is to be understood that the drawings are for purposes of illustrating the concepts of the present disclosure and may not be drawn to scale. Furthermore, the drawings illustrate exemplary embodiments and do not represent limitations to the scope of the present disclosure

### DETAILED DESCRIPTION

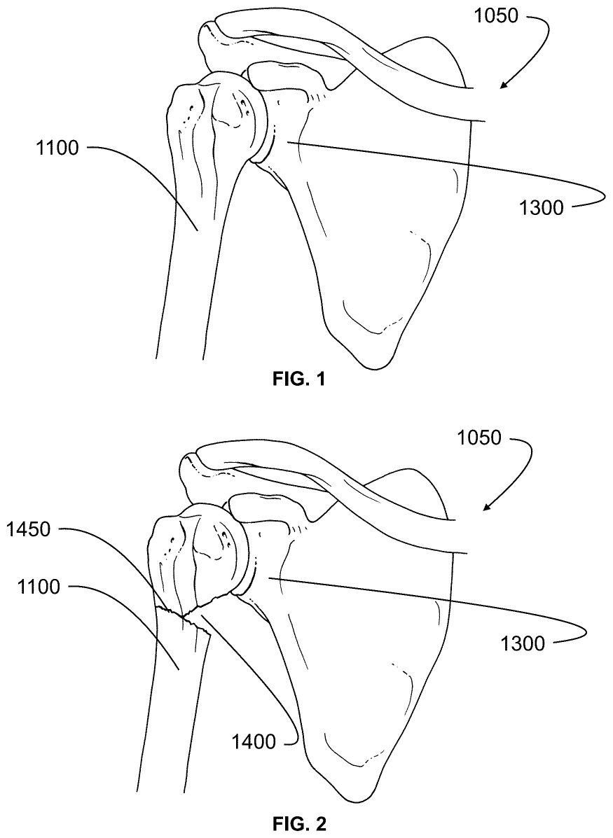

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIG. 1 through FIG. 27B, is not intended to limit the scope of the claims, but is merely representative of exemplary embodiments of the present disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Not every feature of each embodiment is labeled in every figure in which that embodiment appears, in order to keep the figures clear. Similar reference numbers (for example, those that are identical except for the first numeral) may be used to indicate similar features in different embodiments.

The present disclosure relates to adjunctive implants for fracture stabilization for the purposes of illustrating the concepts of the present design. However, it will be understood that other variations and uses are contemplated including, but not limited to, adjunctive implants for compression fractures, osteonecrosis, cancer, joint fusion, joint replacement, partial joint replacement, and/or bone defects.

FIG. 1 is a perspective view of some of the bones generally forming a shoulder joint 1050, including a humerus 1100 and a scapula 1300. FIG. 2 is a perspective view of an exemplary shoulder joint 1050 with an exemplary fracture 1450 of a proximal humerus. The exemplary fracture 1450 may also include a bone void 1400. The bone void 1400 may be located on a medial side of the humerus 1100. The bone void 1400 may result in insufficient structural strength of the bone.

FIG. 3 is a perspective view of an exemplary shoulder joint 1050 with an exemplary fracture 1450 of a proximal humerus and a bone plate 1500. The bone plate 1500 may include a plurality of fasteners 1600 configured to couple the bone plate 1500 to the humerus 1100 and spanning the exemplary fracture 1450. The bone plate 1500 may be configured to reduce the fracture 1450. Fracture reduction is a medical procedure that aims to realign and stabilize fractured bones. The procedure involves manipulating the bone fragments into their proper position and immobilizing them to allow for healing. However, fracture reduction alone may not restore the structural strength of the bone.

The bone plate 1500 may restore alignment of the humerus 1100, and/or reduce the fracture, but may not provide sufficient structural strength to compensate for the bone void 1400. In patients with comminuted complex proximal humerus fractures, using a bone plate alone may not provide stable fixation, and may lead to complications such as varus collapse and/or nonunion.

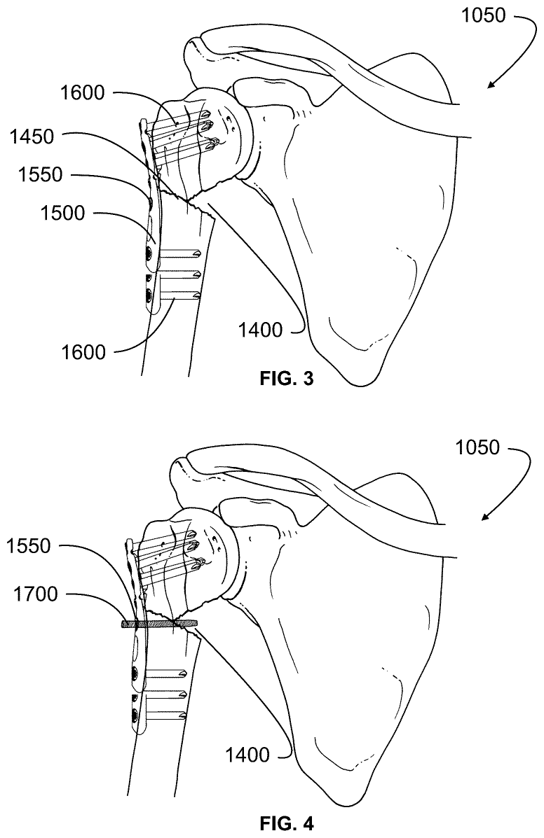

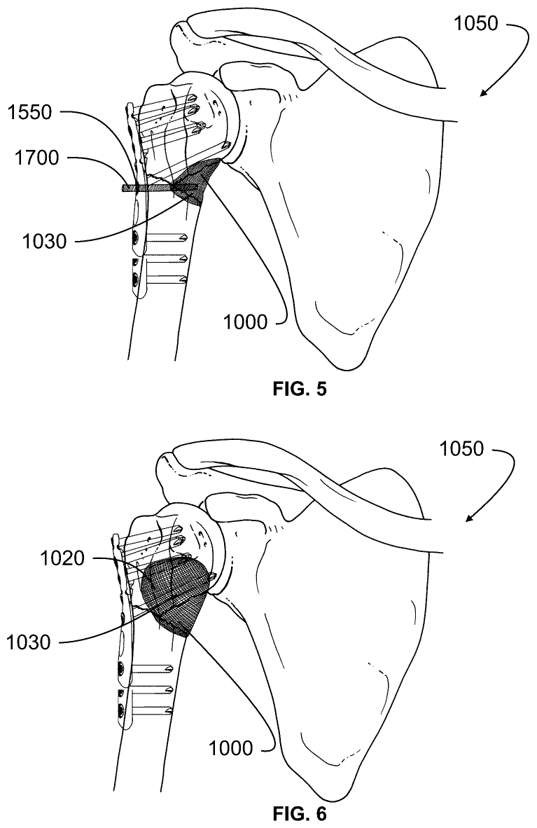

FIG. 4 is a perspective view of an exemplary shoulder joint 1050 with an exemplary fracture 1450 of a proximal humerus, a bone plate 1500, and a cannula 1700 according to an embodiment of the present disclosure. FIG. 5 is a perspective view of an exemplary shoulder joint 1050 with an exemplary fracture 1450 of a proximal humerus, a bone plate 1500, a cannula 1700, and an adjunctive implant 1000 according to an embodiment of the present disclosure. A hole may be drilled in the humerus 1100, creating a tunnel through an access aperture 1550 into the bone void 1400. A cannula 1700 may be configured to be inserted through the access aperture 1550, through the tunnel, and into the bone void 1400.

The adjunctive implant 1000 may be configured to be inserted, in a compressed state, though the cannula 1700 and into the bone void 1400. The cannula 1700 may be further configured to facilitate introduction of a bone filler material 1020 into the interior portion 1030, thereby transitioning the adjunctive implant 1000 from a compressed state to an expanded state. The bone filler material 1020 may be transported though the cannula 1700 and into the interior portion 1030 using a plunger, a tamp, an auger, and/or other methods known in the art. The adjunctive implant may be transitionable from the compressed state to the expanded state through introduction of the bone filler material into the interior portion.

The adjunctive implant 1000 may include an interior portion 1030 configured to receive the bone filler material 1020. The adjunctive implant 1000 may include a compressed state and an expanded state, wherein the adjunctive implant 1000 is transitioned from a compressed state to an expanded state through the introduction of the bone filler material 1020 into the interior portion 1030.

The adjunctive implant 1000 may be configured so that with the adjunctive implant 1000 in the expanded state, the adjunctive implant 1000 takes the shape of the bone void and/or the cavity in which it is received. Additionally, or alternatively, the adjunctive implant 1000 may be configured so that, in the expanded state, the adjunctive implant 1000 generally fills the bone void and/or the cavity in which it is received. Additionally, or alternatively, the adjunctive implant 1000 may be configured so that, in the expanded state, the adjunctive implant 1000 generally conforms to the shape of the bone void and/or the cavity in which it is received.

FIG. 6 is a perspective view of an exemplary shoulder joint 1050 with an exemplary fracture 1450 of a proximal humerus, a bone plate 1500, a cannula 1700, and the adjunctive implant 1000. In the expanded state, the adjunctive implant 1000 may at least partially fill the bone void 1400 and may be proximate the internal surfaces of the humerus 1100 exposed by the bone void 1400. After expansion of the adjunctive implant 1000, the cannula 1700 may be removed from the humerus 1100.

The adjunctive implant 1000 may be configured to complement other orthopedic implants, such as plates, fasteners, and/or nails, in fracture stabilization. The adjunctive implant 1000 may be configured to couple to the first implant. The adjunctive implant 1000 may include an attachment feature configured to be secured to a first implant. Additionally, or alternatively, the adjunctive implant 1000 may be configured to receive a fastener, such as a screw, a pin, a nail, and/or another orthopedic fastener known in the art. The fastener may couple the adjunctive implant 1000 to the first implant.

Additionally, or alternatively, the adjunctive implant 1000 may act as an internal cast to provide structural support of a fracture and/or a bone portion. The adjunctive implant 1000 may include a bioabsorbable material configured to be absorbed into the surrounding bone tissues. Additionally, or alternatively, the adjunctive implant 1000 may include a porous material configured to facilitate bone growth through the adjunctive implant 1000.

The adjunctive implant 1000 may be manufactured using bioabsorbable silk and/or biocompatible silk. Additionally, or alternatively, the adjunctive implant 1000 may be manufactured using synthetic bioabsorbable polyglycolide-trimethylene carbonate copolymer (PGA-TMC), synthetic fully resorbable poly-4-hydroxybutyrate (P4HB), other synthetic bioabsorbable polymers, and/or natural bioabsorbable mesh materials. Additionally, or alternatively, the adjunctive implant 1000 may be manufactured using an autograft and/or an allograft material, such as fetal material/tissue, placental material/tissue, umbilical material/tissue, and/or another suitable material/tissue.

The bioabsorbable and/or biocompatible silk may be configured to have both soft, malleable portions as well as rigid structural portions in the same adjunctive implant 1000. For example, the adjunctive implant 1000 may be configured as a rigid silk device with a malleable silk balloon portion wrapped around it, whereby the malleable silk balloon may be filled with graft material. The fully deployed adjunctive implant 1000 may have both structural rigidity from the rigid silk portion as well as mechanical rigidity from the malleable silk balloon containing graft material filling a bone void 2300. A bone void 2300 may be defined as a void within a single bone due to a fracture, a trauma, a defect, a tumor, an infection, a congenital defect, and/or another etiology.

The adjunctive implant 1000 may be manufactured with variations in wall thickness configured to provide variations in strength, bio-absorption, and/or geometry. The adjunctive implant 1000 may be configured so that, in the expanded state, the adjunctive implant 1000 is generally shaped as a sphere, a cube, a cylinder, a pyramid, a cone, a horn, or a combination of two or more shapes. Additionally, or alternatively, the adjunctive implant 1000 may be manufactured with geometry based on specific patient anatomy and/or fracture parameters. Additionally, or alternatively, the adjunctive implant 1000 may be configured so that, regardless of the manufactured shape, the adjunctive implant 1000 may conform to the shape of the bone void 2300 when in the expanded state.

The adjunctive implant 1000 may be configured to receive bone filler material 1020 within the interior portion 1030. The bone filler material may be one of, or a combination of: demineralized bone matrix, allograft bone graft, autograft bone graft, autologous bone graft, bone cement, hydroxyapatite, granular bone graft, tricalcium phosphate, calcium sulfate, calcium phosphate, bone marrow aspirate, antibiotic-impregnated calcium sulfate, allopathic bioactive molecules, xenomorphic bioactive molecules, synthetic bioactive molecules, osteoconductive synthetic polymers, and/or other biologic adjuvants configured to provide an osteoconductive matrix to facilitate proliferation of bone.

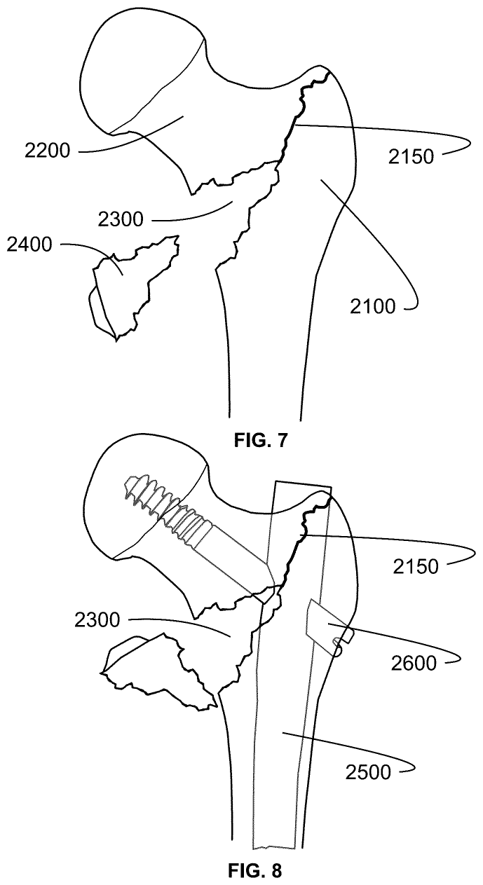

FIG. 7 is a perspective view of an exemplary femur 2100 with an exemplary fracture 2150 and bone fragment 2400. FIG. 8 is a perspective view of an exemplary femur 2100 with an exemplary fracture 2150 and bone fragment 2400 and an exemplary intramedullary nail 2500 and fastener 2600. The exemplary fracture 2150 may also include a bone void 2300. The bone void 2300 may be located on a medial side of the femur 2100. The intramedullary nail 2500 and fastener 2600 may restore alignment of the femur 2100 and femoral neck 2200, but may not provide sufficient structural strength to compensate for the bone void 2300. In patients with complex proximal femoral fractures, using an intramedullary nail and/or fastener alone may not provide stable fixation, and may lead to complications such as varus collapse and/or nonunion.

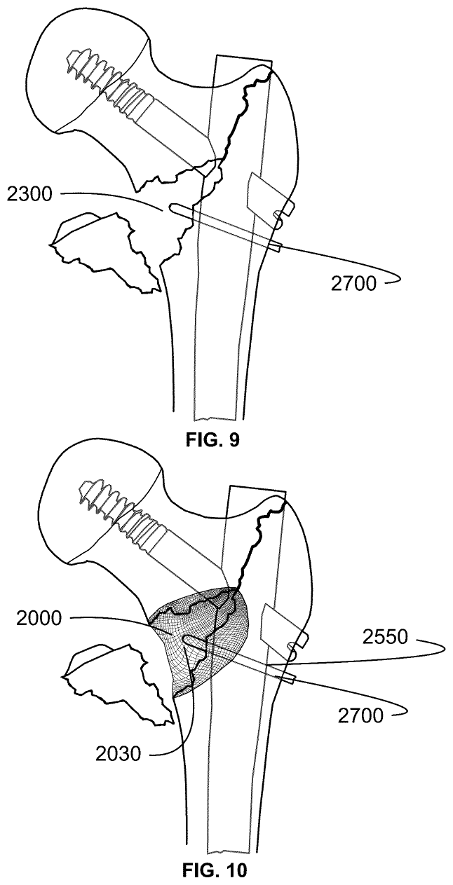

FIG. 9 is a perspective view of an exemplary femur 2100 with an exemplary fracture 2150 and bone fragment 2400, an exemplary intramedullary nail 2500 and fastener 2600, and a cannula 2700 according to an embodiment of the present disclosure. FIG. 10 is a perspective view of an exemplary femur 2100 with an exemplary fracture 2150 and bone fragment 2400, an exemplary intramedullary nail 2500 and fastener 2600, a cannula 2700, and an adjunctive implant 2000 according to an embodiment of the present disclosure. The adjunctive implant 2000 may incorporate all features and properties of the adjunctive implant 1000 previously described. The adjunctive implant 2000 may be configured to provide fracture reduction, fracture stabilization, and/or structural support to a bone, in conjunction with a first implant.

A hole may be drilled in the femur 2100, creating a tunnel 2040 (as shown in FIG. 12) through a nail aperture 2550 into the bone void 2300. A cannula 2700 may be configured to be inserted through the nail aperture 2550, through the tunnel 2040, and into the bone void 2300. The adjunctive implant 2000 may include an interior portion 2030 configured to receive a bone filler material 2020. The adjunctive implant 2000 may include a compressed state and an expanded state, wherein the adjunctive implant 2000 is transitioned from a compressed state to an expanded state through the introduction of the bone filler material 2020 into the interior portion 2030.

The adjunctive implant 2000 may be configured to be insertable, in a compressed state, though the cannula 2700 and into the bone void 2300. The cannula 2700 may be further configured to facilitate introduction of bone filler material 2020 into the interior portion 2030, thereby transitioning the adjunctive implant 2000 from a compressed state to an expanded state. The bone filler material 2020 may be transported though the cannula 2700 and into the interior portion 2030 using a plunger, a tamp, an auger, and/or other methods known in the art.

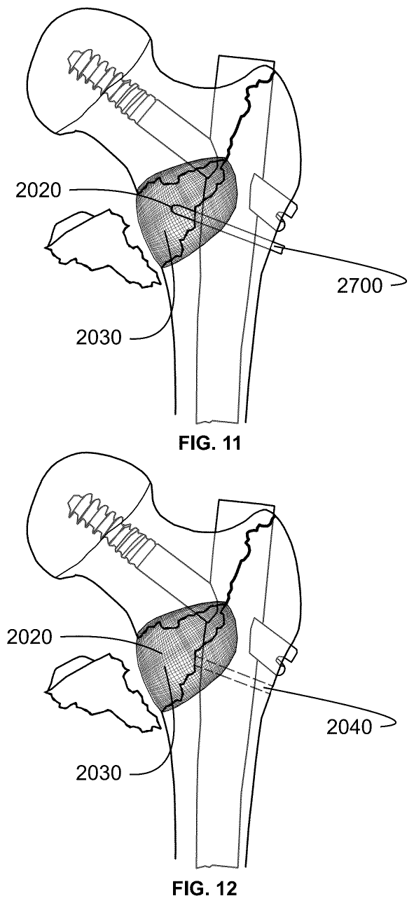

FIG. 11 is a perspective view of an exemplary femur 2100 with an exemplary fracture 2150 and bone fragment 2400, an exemplary intramedullary nail 2500 and fastener 2600, a cannula 2700, and the adjunctive implant 2000. FIG. 12 is a perspective view of an exemplary femur 2100 with an exemplary fracture 2150 and bone fragment 2400, an exemplary intramedullary nail 2500 and fastener 2600, and the adjunctive implant 2000. In the expanded state, the adjunctive implant 2000 may at least partially fill the bone void 2300 and may be proximate the internal surfaces of the femur 2100 exposed by the bone void 2300. After expansion of the adjunctive implant 2000, the cannula 2700 may be removed from the femur 2100.

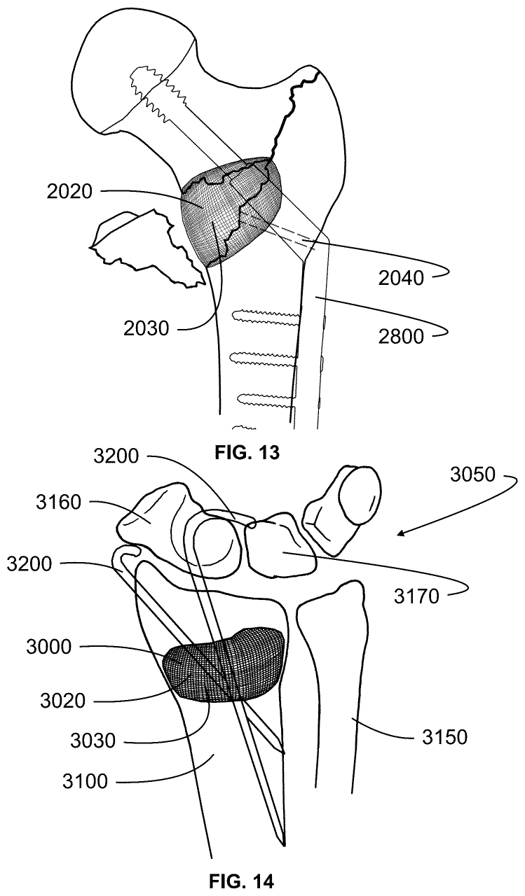

FIG. 13 is a perspective view of an exemplary femur 2100 with an exemplary fracture 2150 and bone fragment 2400, a bone plate 2800, and an adjunctive implant 2000 according to an embodiment of the present disclosure. The adjunctive implant 2000 may be configured to receive a portion of the bone plate 2800 thereby coupling the bone plate 2800 to the adjunctive implant 2000 and limiting migration and/or subsidence of the adjunctive implant 2000. The adjunctive implant 2000 may be expanded within the bone void 2300 after fracture reduction but prior to placement of the bone plate 2800. Alternatively, the bone plate 2800 may be placed to reduce the fracture and then the adjunctive implant 2000 may be expanded within the bone void 2300.

FIG. 14 is a perspective view of some of the bones generally forming a wrist 3050, pins 3200, and an adjunctive implant 3000 according to an embodiment of the present disclosure. The adjunctive implant 3000 may incorporate all features and properties of the adjunctive implant 1000 and the adjunctive implant 2000 previously described. The wrist 3050 may generally include a distal radius 3100, a distal ulna 3150, a scaphoid 3160, a lunate 3170, and other bones (not shown). Surgical treatment of fractures of the distal radius 3100 may involve implantation of bone filler material 3020 into bone voids that may arise after fracture reduction. The adjunctive implant 3000 may be configured to fill a bone void within a distal radius 3100.

The adjunctive implant 3000 may be configured to receive one or more pins 3200. The one or more pins 3200 may limit migration and/or subsidence of the adjunctive implant 3000 within the distal radius 3100. The adjunctive implant 3000 may include an interior portion 3030 configured to receive a bone filler material 3020. The adjunctive implant 3000 may include a compressed state and an expanded state, wherein the adjunctive implant 3000 is transitioned from a compressed state to an expanded state through the introduction of the bone filler material 3020 into the interior portion 3030.

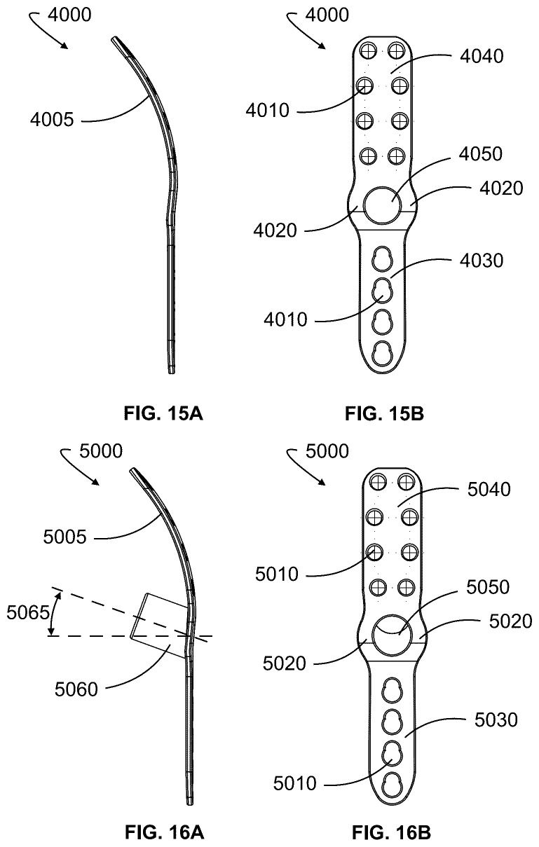

FIG. 15A is a side view of a bone plate 4000 according to an embodiment of the present disclosure. FIG. 15B is a front view of the bone plate 4000. Various parts of the bone plate 4000 may be identical or similar to their counterparts on the bone plate 1500 and/or other bone plate embodiments presented herein; these parts may not be described again here. All statements made regarding the bone plate 1500 apply to the bone plate 4000 unless they would be contradicted by the differences between the two.

The bone plate 4000 may be configured to reduce a fracture a fracture of a bone of a patient. Additionally, or alternatively, the bone plate 4000 may be configured to stabilize a fracture and/or a bone void of a humerus. In other embodiments the bone plate 4000 may be configured to reduce and/or stabilize a fracture of a femur, a wrist bone, a foot bone, and/or other joint adjacent bone of a patient.

The bone plate 4000 may include a bone facing side 4005, a plurality of fastener apertures 4010, a reinforced central portion 4020, an inferior portion 4030, a superior portion 4040, and an access aperture 4050. The bone plate 4000 may be secured to an exterior portion of a humerus of a patient such that the bone facing side 4005 contact the exterior of the humerus. The bone plate 4000 may be positioned, relative to the humerus, so that the inferior portion 4030 is directed inferiorly and the superior portion 4040 is directed superiorly. Each of the inferior portion 4030 and the superior portion 4040 may include a plurality of fastener apertures 4010. Each of the fastener apertures may be configured to receive a fastener (not shown) configured to secure the bone plate 4000 to the humerus.

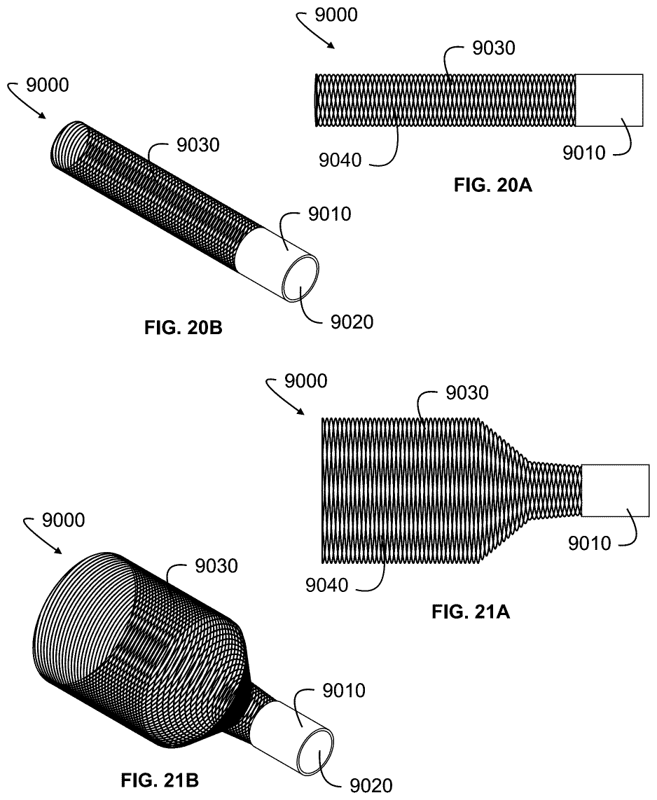

The access aperture 4050 may be located between the inferior portion 4030 and the superior portion 4040. The access aperture 4050 may be larger than each of the fastener apertures 4010. The access aperture 4050 may be sized to receive a cannula, such as cannula 1700, and/or an adjunctive implant, such as an adjunctive implant 9000 as shown in FIG. 20A. Additionally, or alternatively, the access aperture 4050 may be sized to receive a support device, such as: a bone graft, a bone dowel, a graft containment device, a polymer bone filler, a reinforcement implant, and/or another support device.

The access aperture 4050 may be configured so that the adjunctive implant 9000, and/or the support device may be coupled with the bone plate 4000, thereby providing integration of the bone plate 4000 and the support device.

The reinforced central portion 4020 may circumscribe the access aperture 4050 to provide adequate fatigue strength of the bone plate 4000. The reinforced central portion 4020 may extend beyond an outer profile of the inferior portion 4030 and/or the superior portion 4040.

FIG. 16A is a side view of a bone plate 5000 according to an embodiment of the present disclosure. FIG. 16B is a front view of the bone plate 5000. Various parts of the bone plate 5000 may be identical or similar to their counterparts on the bone plate 4000 and/or other bone plate embodiments presented herein; these parts may not be described again here. All statements made regarding the bone plate 4000 apply to the bone plate 5000 unless they would be contradicted by the differences between the two.

The bone plate 5000 may include a bone facing side 5005, a plurality of fastener apertures 5010, a reinforced central portion 5020, an inferior portion 5030, a superior portion 5040, an access aperture 5050, a fixed angle protrusion 5060, and a protrusion angle 5065.

The fixed angle protrusion 5060 may extend from the bone facing side 5005 at the protrusion angle 5065. The protrusion angle 5065 may be between 10 degrees and 30 degrees. More specifically, the protrusion angle may be 20 degrees. The bone plate 5000 may be one of a set of differently-sized bone plates, each having a different protrusion angle 5065.

The fixed angle protrusion 5060 may be configured to be received in a bone tunnel. The bone tunnel may extend from an exterior surface of the bone to a bone void. The access aperture 5050 may extend through the fixed angle protrusion 5060 so that the adjunctive implant 9000 and/or other support device may be received in the access aperture so that the adjunctive implant 9000 and/or the other support device may be received within the bone void.

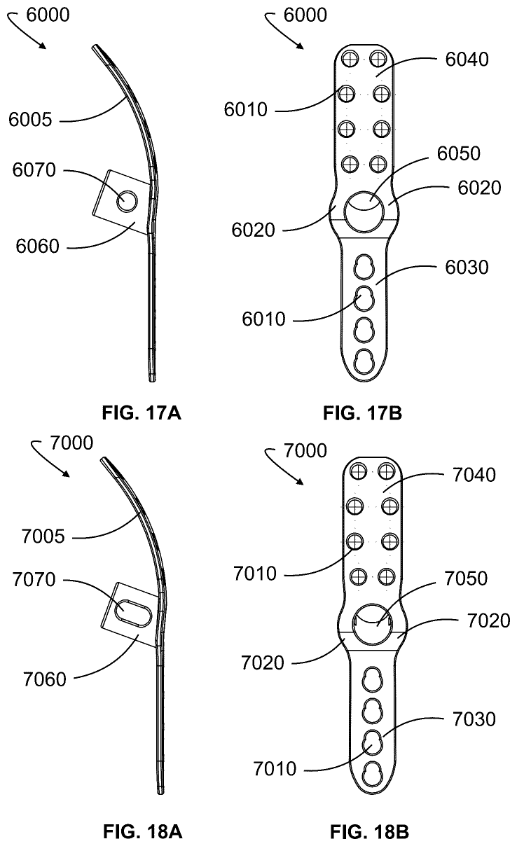

FIG. 17A is a side view of a bone plate 6000 according to an embodiment of the present disclosure. FIG. 17B is a front view of the bone plate 6000. Various parts of the bone plate 6000 may be identical or similar to their counterparts on the bone plate 5000 and/or other bone plate embodiments presented herein; these parts may not be described again here. All statements made regarding the bone plate 5000 apply to the bone plate 6000 unless they would be contradicted by the differences between the two.

The bone plate 6000 may include a bone facing side 6005, a plurality of fastener apertures 6010, a reinforced central portion 6020, an inferior portion 6030, a superior portion 6040, an access aperture 6050, a fixed angle protrusion 6060, and a protrusion aperture 6070.

The protrusion aperture 6070 may extend generally perpendicular to a longitudinal axis of the fixed angle protrusion 6060. The protrusion aperture 6070 may extend through both walls of the fixed angle protrusion 6060. The protrusion aperture 6070 may be generally circular in shape and may be sized to receive a fastener (not shown). The fastener may be advanced through the protrusion aperture to secure the adjunctive implant 9000 and/or other support device to the bone plate 6000. Additionally, or alternatively, the fastener may be advanced through the bone and into the protrusion aperture to help secure the bone plate 6000 to the bone.

FIG. 18A is a side view of a bone plate 7000 according to an embodiment of the present disclosure. FIG. 18B is a front view of the bone plate 7000. Various parts of the bone plate 7000 may be identical or similar to their counterparts on the bone plate 6000 and/or other bone plate embodiments presented herein; these parts may not be described again here. All statements made regarding the bone plate 6000 apply to the bone plate 7000 unless they would be contradicted by the differences between the two.

The bone plate 7000 may include a bone facing side 7005, a plurality of fastener apertures 7010, a reinforced central portion 7020, an inferior portion 7030, a superior portion 7040, an access aperture 7050, a fixed angle protrusion 7060, and a protrusion aperture 7070.

The protrusion aperture 7070 may extend generally perpendicular to a longitudinal axis of the fixed angle protrusion 7060. The protrusion aperture 7070 may extend through both walls of the fixed angle protrusion 7060. The protrusion aperture 7070 may be generally elongate in shape and may be sized to receive a fastener (not shown) in a plurality of locations. The fastener may be advanced through the protrusion aperture to secure the adjunctive implant 9000 and/or other support device to the bone plate 7000. Additionally, or alternatively, the fastener may be advanced through the bone and into the protrusion aperture to help secure the bone plate 7000 to the bone.

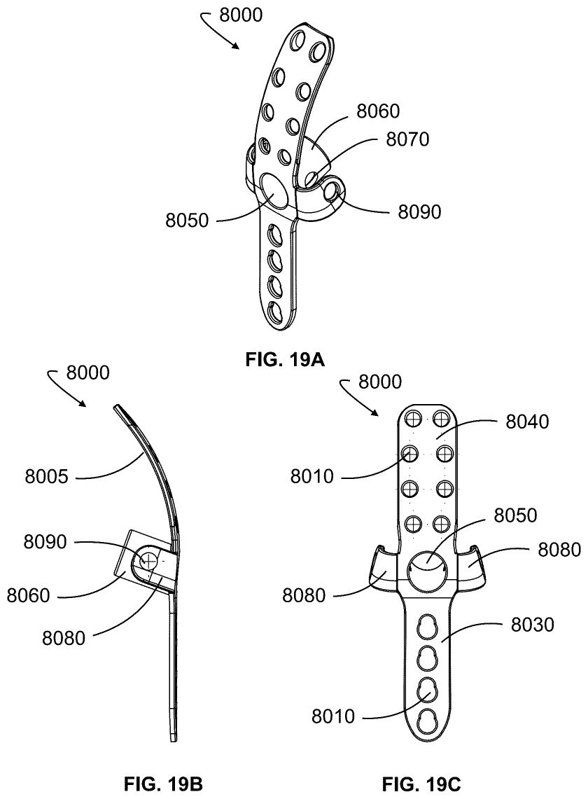

FIG. 19A is a perspective view of a bone plate 8000 according to an embodiment of the present disclosure. FIG. 19B is a side view of the bone plate 8000 and FIG. 19C is a front view of the bone plate 8000. Various parts of the bone plate 8000 may be identical or similar to their counterparts on the bone plate 6000 and/or other bone plate embodiments presented herein; these parts may not be described again here. All statements made regarding the bone plate 6000 apply to the bone plate 8000 unless they would be contradicted by the differences between the two.

The bone plate 8000 may include a bone facing side 8005, a plurality of fastener apertures 8010, an inferior portion 8030, a superior portion 8040, an access aperture 8050, a fixed angle protrusion 8060, a protrusion aperture 8070, support wings 8080, and wing apertures 8090.

The support wings 8080 may extend bilaterally from the bone plate 8000 proximate the access aperture 8050. The support wings 8080 may further extend towards the bone facing side 8005 of the bone plate 8000. Each of the support wings 8080 may include one or more wing apertures 8090. The support wings 8080 may be configured to engage an exterior surface of the bone. The support wings 8080 may be malleable so that they may be bent and/or adjusted to conform to the exterior surface of the bone. The wing apertures 8090 may be generally circular in shape and may be sized to receive a fastener (not shown).

The wing apertures 8090 may generally align with the protrusion apertures 8070. The fastener may be advanced through the wing aperture 8090 and the protrusion apertures 8070 to secure the adjunctive implant 9000 and/or other support device to the bone plate 8000. Additionally, or alternatively, the fastener may be advanced through wing aperture 8090 and into the bone to help secure the bone plate 8000 to the bone.

FIG. 20A is a front view of an expandable adjunctive implant 9000 in a compressed state according to an embodiment of the present disclosure. FIG. 20B is a perspective view of the expandable adjunctive implant 9000 in the compressed state. FIG. 21A is a front view of the expandable adjunctive implant 9000 in an expanded state according to an embodiment of the present disclosure. FIG. 21B is a perspective view of the expandable adjunctive implant 9000 in the expanded state.

The adjunctive implant 9000 may include a compressed state and an expanded state. The adjunctive implant 9000 may be configured to be received within the bone void such that the adjunctive implant 9000 may provide structural support to the bone from within the bone void. In the expanded state, the adjunctive implant 9000 may generally conform to a shape of the bone void. In the compressed state, the adjunctive implant 9000 may be insertable through the access aperture 8050 into the bone void. In the compressed state, the adjunctive implant 9000 may be insertable through a cannula into the bone void.

The adjunctive implant 9000 may include a bioabsorbable material configured to be absorbed by the patient. The adjunctive implant 9000 may include a malleable silk balloon configured to receive a bone filler material. Additionally, or alternatively, the adjunctive implant 9000 may include an autograft and/or an allograft material, such as fetal material/tissue, placental material/tissue, umbilical material/tissue, and/or another suitable material/tissue.

The adjunctive implant 9000. The adjunctive implant may include an interior portion 9040 configured to receive the bone filler material so that the adjunctive implant 9000 may be transitionable from the compressed state to the expanded state through introduction of the bone filler material into the interior portion.

The adjunctive implant 9000 may further include an expandable portion 9030 and a base portion 9010 including a base aperture 9020. The expandable portion 9030 may extend from the base aperture 9020. The expandable portion 9030 may include the interior portion 9040. The expandable portion 9030 may be configured to expand in response to introduction of a bone filler material into the interior portion 9040. The expandable portion 9030 may be configured to conform to a shape of a cavity in which the expandable portion 9030 is expanded. The expandable portion 9030 may be configured as a mesh, a knitted material, a woven material, an expandable material, and/or a non-expandable material that is manufactured with an expandable construction.

The base portion 9010 may be configured to be received in the access aperture 8050. The base aperture 9020 may extend to the interior portion 9040 so that a bone filler material may be received in the base aperture 9020 and introduced into the interior portion 9040. The base portion 9010 may include a coupling feature to securely couple the adjunctive implant 9000 to the bone plate 8000, or more specifically, the base portion 9010 to the fixed angle protrusion 8060. The coupling feature may include a taper lock, a threaded portion, a spline, a j-lock, tabs, a collet, a locking faster, or other features that securely couple the base portion 9010 to the fixed angle protrusion 8060. Additionally, or alternatively, the adjunctive implant 9000 may be couple to the bone plate 8000 using a fastener that engages both the adjunctive implant 9000 and the bone plate 8000.

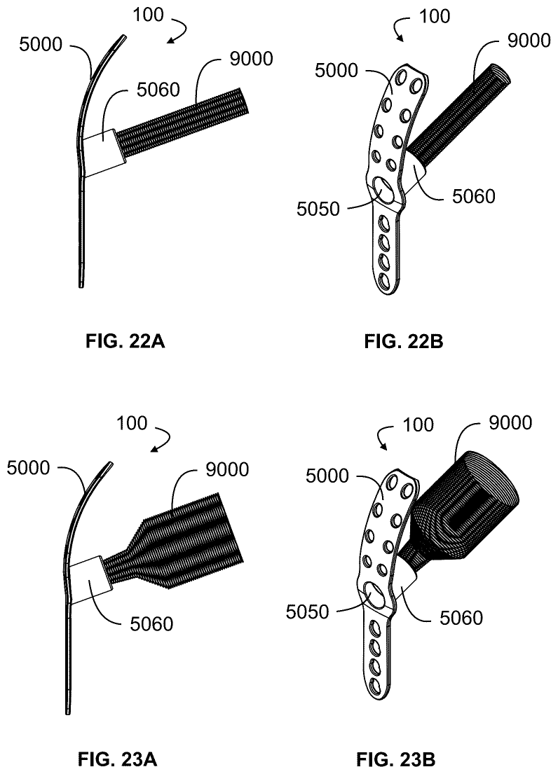

FIG. 22A is a side view of a system for stabilization 100 of a bone of a patient including the expandable adjunctive implant 9000 and the bone plate 5000 with the expandable adjunctive implant 9000 in a compressed state according to an embodiment of the present disclosure. FIG. 22B is a perspective view of the system for stabilization 100 of a bone of a patient in the compressed state. FIG. 23A is a side view of a system for stabilization 100 of a bone of a patient including the expandable adjunctive implant 9000 and the bone plate 5000 with the expandable adjunctive implant 9000 in an expanded state according to an embodiment of the present disclosure. FIG. 23B is a perspective view of the system for stabilization 100 of a bone of a patient in the expanded state.

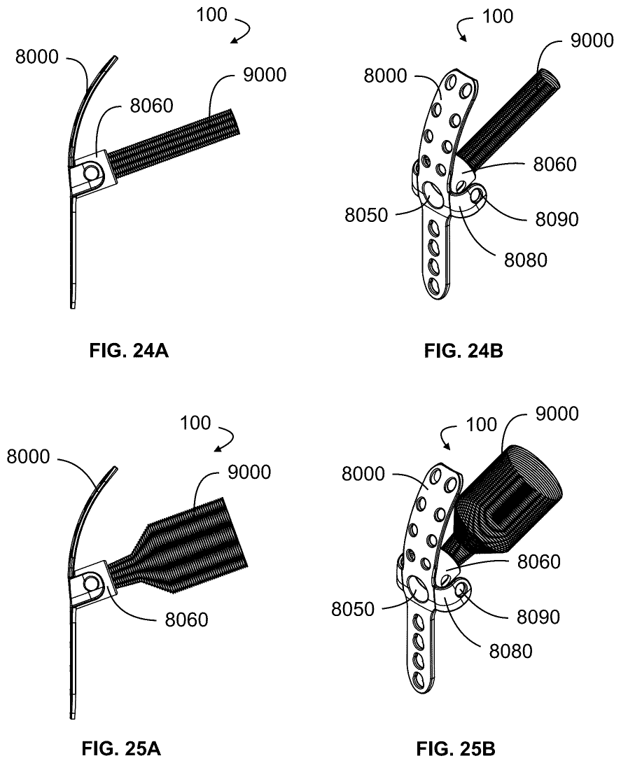

FIG. 24A is a side view of a system for stabilization 100 of a bone of a patient including the expandable adjunctive implant 9000 and the bone plate 8000 with the expandable adjunctive implant 9000 in a compressed state according to an embodiment of the present disclosure. FIG. 24B is a perspective view of the system for stabilization 100 of a bone of a patient in the compressed state. FIG. 25A is a side view of a system for stabilization 100 of a bone of a patient including the expandable adjunctive implant 9000 and the bone plate 8000 with the expandable adjunctive implant 9000 in an expanded state according to an embodiment of the present disclosure. FIG. 25B is a perspective view of the system for stabilization 100 of a bone of a patient in the expanded state.

The system for stabilization 100 may be a unified implant including a bone plate, such as bone plate 5000, bone plate 6000, bone plate 7000, and/or bone plate 8000 manufactured jointly with an adjunctive implant, such as the adjunctive implant 9000. The system for stabilization 100 may be additively manufactured as a unified implant.

In an embodiment, the system for stabilization 100 may be additively manufactured using a biocompatible material. Additionally, or alternatively, the system for stabilization 100 may be manufactured using a bioabsorbable material configured to be absorbed by the patient. Additionally, or alternatively, the system for stabilization 100 may include a surface coating to enhance biocompatibility and/or promote osteointegration.

Additionally, or alternatively, the system for stabilization 100 may be additively manufactured using Tullomer™ filament, an ultra-fine monofilament polymer fiber, silk, and/or a standard polymer filament. The system for stabilization 100 may be additively manufactured using standard additive manufacturing technology, such as: stereolithography (SLA), fused deposition modeling (FDM), binder jetting (BJT), digital light processing (DLP), and/or another additive manufacturing technology known in the art.

The system for stabilization 100 may be configured to stabilize a fracture and a bone void of a bone, such as a humerus, of a patient. The system for stabilization 100 may include a first segment configured to reduce a fracture of a bone, such as a humerus, and a second segment configured to be coupled with the first segment. The first segment may include a first material and the second segment may include a second material. The first material may be different than the second material. The first material may be more rigid than the second material. The second material may be more flexible than the first material. The first material and/or the second material may include a bioabsorbable material configured to be absorbed by a patient.

Alternatively, the first material and the second material may be the same material. Additionally, or alternatively, the first material and the second material may include different mechanical properties. Additionally, or alternatively, the system for stabilization 100 may be configured with the first material the same as the second material, but, through an engineered structure that may be generated through the additive manufacturing process, the first material may have different mechanical properties than the second material.

FIG. 26A is a side view of a system for stabilization 100 of a bone of a patient including the expandable adjunctive implant 9000 and the bone plate 8000 with the expandable adjunctive implant 9000 in a compressed state and the system in a pre-coupled state according to an embodiment of the present disclosure. FIG. 26B is a perspective view of the system for stabilization 100 of a bone of a patient in the pre-coupled state.

The system for stabilization 100 may include a bone plate, such as bone plate 5000, bone plate 6000, bone plate 7000, and/or bone plate 8000 couplable to an adjunctive implant, such as the adjunctive implant 9000. The bone plate 8000 may further include a distal side 8092 and a proximal side 8094. The distal side 8092 may be the bone facing side 8005. The proximal side 8094 may be opposite the distal side 8092.

The adjunctive implant 9000 may be received in the bone plate 8000, more specifically, the base portion 9010 may be received in the distal side 8092 of the fixed angle projection 8060. This may allow the adjunctive implant 9000 to be coupled to the bone plate 8000 prior to insertion of the adjunctive implant into a bone tunnel. The coupling feature may include a taper lock, a threaded portion, a spline, a j-lock, tabs, a collet, a locking faster, or other features that securely couple the base portion 9010 to the fixed angle protrusion 8060. With the adjunctive implant 9000 coupled to the bone plate 8000, the access aperture 8050 may align with the base aperture 9020, thereby allowing introduction of bone filler material through the access aperture 8050, through the base aperture 9020, and into the interior portion 9040 to transition the adjunctive implant 9000 from the collapsed state to the expanded state.

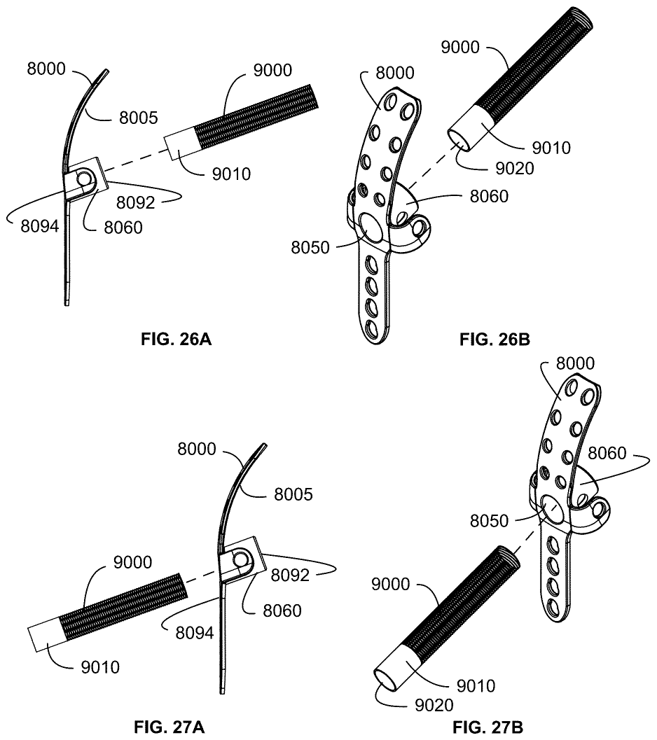

FIG. 27A is a side view of a system for stabilization 100 of a bone of a patient including the expandable adjunctive implant 9000 and the bone plate 8000 with the expandable adjunctive implant 9000 in a compressed state and the system in a pre-coupled state according to an embodiment of the present disclosure. FIG. 27B is a perspective view of the system for stabilization 100 of a bone of a patient in the pre-coupled state.

The adjunctive implant 9000 may be received in the bone plate 8000, more specifically, the base portion 9010 may be received in the proximal side 8094 of the fixed angle projection 8060. This may allow the adjunctive implant 9000 to be coupled to the bone plate 8000 after the bone plate 8000 has been secured to the bone. With the bone plate 8000 secured to the bone, the fixed angle protrusion 8060 may be received in a bone tunnel. The coupling feature may include a taper lock, a threaded portion, a spline, a j-lock, tabs, a collet, a locking faster, or other features that securely couple the base portion 9010 to the fixed angle protrusion 8060. The adjunctive implant 9000 may be configured so that, in the collapsed state, the expandable portion 9030 is sized to be received in, and able to be pass through, the access aperture 8050. The adjunctive implant 9000 may then be pass through a bone tunnel so that at least a portion of the expandable portion 9030 is received in a bone void.

With the adjunctive implant 9000 coupled to the bone plate 8000, the access aperture 8050 may align with the base aperture 9020, thereby allowing introduction of bone filler material through the access aperture 8050, through the base aperture 9020, and into the interior portion 9040 to transition the adjunctive implant 9000 from the collapsed state to the expanded state.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

What is claimed is:

1. A system for stabilization of a bone of a patient, the bone comprising a fracture and a bone void, wherein the bone void results in insufficient structural strength of the bone, the system comprising:
a first implant configured to reduce the fracture of the bone; and
an adjunctive implant configured to be coupled with the first implant;
wherein the adjunctive implant is further configured to be received within the bone void such that the adjunctive implant provides structural support to the bone from within the bone void.

2. The system of claim 1, wherein the adjunctive implant further comprises a bioabsorbable material configured to be absorbed by the patient.

3. The system of claim 1, wherein the adjunctive implant comprises a malleable silk balloon configured to receive a bone filler material.

4. The system of claim 1, wherein the adjunctive implant comprises a compressed state and an expanded state, wherein, in the expanded state, the adjunctive implant generally conforms to a shape of the bone void.

5. The system of claim 4, wherein the system further comprises a cannula configured to receive the adjunctive implant in the compressed state, wherein, with the adjunctive implant in the compressed state, the adjunctive implant is insertable through the cannula into the bone void.

6. The system of claim 4, wherein:
the system further comprises a bone filler material configured to be received within the adjunctive implant;
the adjunctive implant further comprises an interior portion; and
the adjunctive implant is transitionable from the compressed state to the expanded state through introduction of the bone filler material into the interior portion.

7. The system of claim 1, wherein the first implant and the adjunctive implant are additively manufactured as a unified implant.

8. A system for stabilization of a bone of a patient, the bone comprising a fracture and a bone void, the system comprising:
a first implant configured to reduce the fracture of the bone, the first implant comprising an access aperture;
an adjunctive implant comprising a compressed state and an expanded state; and
a bone filler material configured to be introduced into the adjunctive implant;
wherein:
the adjunctive implant is insertable through the access aperture into the bone void;
the bone filler material is insertable through the access aperture into the adjunctive implant; and
the adjunctive implant is transitionable from the compressed state to the expanded state through introduction of the bone filler material.

9. The system of claim 8, wherein, with the adjunctive implant in the expanded state, the adjunctive implant provides structural support to the bone.

10. The system of claim 8, wherein the adjunctive implant is configured to be coupled with the first implant.

11. The system of claim 8, wherein the adjunctive implant further comprises a bioabsorbable material configured to be absorbed by the patient.

12. The system of claim 8, further comprising a cannula insertable through the access aperture and configured to receive the adjunctive implant in the compressed state, wherein, with the adjunctive implant in the compressed state, the adjunctive implant is insertable through the cannula into the bone void.

13. The system of claim 12, wherein the bone filler material is insertable through the cannula into the adjunctive implant.

14. The system of claim 8, wherein, with the adjunctive implant in the expanded state, the adjunctive implant generally conforms to a shape of the bone void.

15. A system for stabilization of a bone of a patient, the bone comprising a fracture and a bone void, the system comprising:
a first segment configured to reduce the fracture of the bone; and
a second segment configured to be coupled with the first segment;
wherein:
the first segment and the second segment are additively manufactured;
the first segment comprises a first material;
the second segment comprises a second material, different than the first material; and
the first material is more rigid than the second material.

16. The system of claim 15, wherein at least one of the first material and the second material comprise a bioabsorbable material configured to be absorbed by the patient.

17. The system of claim 15, wherein at least one of the first segment and the second segment comprises a surface coating configured to promote osteointegration.

18. The system of claim 15, wherein the system is configured to stabilize the fracture and the bone void of a humerus.

19. The system of claim 15, wherein the second segment is configured to be received within the bone void.

20. The system of claim 15, wherein the system further comprises a bone filler material configured to be received within the second segment, and the second segment is transitionable from a compressed state to an expanded state through introduction of the bone filler material.
